(19) United States
(12) Patent Application Publication
(10) Pub. No.: US 2016/0045707 A1
(43) Pub. Date: Feb. 18, 2016

(54) Multiple Side Hole Infusion Apparatus

(71) Applicant: Ernest A. Conti, Crystal Lake, IL (US)

(72) Inventor: Ernest A. Conti, Crystal Lake, IL (US)

(21) Appl. No.: 14/822,288

(22) Filed: Aug. 10, 2015

Related U.S. Application Data
(60) Provisional application No. 62/037,903, filed on Aug. 15, 2014.

(51) Int. Cl. A61M 25/00; A61M 25/01; A61M 5/14

(52) U.S. Cl. CPC A61M 25/007; A61M 5/1407; A61M 25/0043; A61M 25/01; A61M 25/0084; A61M 2025/0073; A61M 2025/0057; A61M 2025/0063; A61M 2025/0092; A61M 2025/018

(57) ABSTRACT

A multiple side hole infusion apparatus is a device that is used to deliver and distribute medical fluid over a targeted area as well as facilitate the insertion of a guide wire or similar object into a patient's body. The device features a tubular infusion member that is inserted into and able to slide within a tubular sleeve member. A quantity of dynamic fluid is introduced into the tubular infusion member and is able to exit through a fluid-release port located on the tubular infusion member and into an annular clearance between the tubular infusion member and the tubular sleeve member. The quantity of dynamic fluid is then able to exit the tubular sleeve member through a plurality of fluid-distribution ports that is located along the length of the tubular sleeve member, enabling the quantity of dynamic fluid to spread over a targeted area within the patient's body.

The current application claims a priority to the U.S. Provisional Patent application Ser. No. 62/037,903 filed on Aug. 15, 2014.

### FIELD OF THE INVENTION

The present invention relates generally to a medical fluid infiltration and infusion device. More specifically, the present invention is a multiple side hole infusion apparatus that facilitates the infiltration or infusion of medical fluid into a patient's body over a wide area surrounding the apparatus within soft tissues or blood vessels.

### BACKGROUND OF THE INVENTION

When performing invasive surgical procedures such as abscess drainage, biliary intervention, and percutaneous nephrostomy drain placement, a hollow needle is initially inserted into the patient's body in order to remove fluid or air from the patient's body. During these invasive surgical procedures, a thin needle is inserted into the patient's body and advanced to the area within the patient's body from which fluid or air must be removed. A rigid wire is then inserted through the thin needle, with the wire left in place within the patient's body in order to serve as a guide for a second, larger drain cannula or catheter that is subsequently advanced over the guide wire into the patient's body to the fluid or air removal site. The guide wire is removed after the drain cannula or catheter is in place within the patient's body. The drain cannula or catheter is then used to remove the targeted fluid or air.

Insertion of a large foreign object such as a drain cannula or catheter through multiple deep tissue layers can be quite painful and as such, the operating medical professional generally utilizes a generous volume of local anesthetic in order to alleviate pain experienced by the patient during the invasive procedure. During anesthetization, the skin surface is first anesthetized in order to numb the majority of the pain nerves at the skin surface. Anesthetics are then injected into the deep tissues around the needle tract in an attempt to further alleviate pain through the deep tissue layers. However, because it is not possible to see under the skin surface, it is difficult to accurately anesthetize the deep tissues along the needle tract. Consequently, it is common for the patient to feel pain in the surrounding deep tissues when the cannula or catheter is advanced over the guide wire.

The present invention is a multiple side hole infusion apparatus that is designed to more effectively distribute and increase the effectiveness of a fluid such as a local anesthetic that is injected into the deep tissues at the procedure site. The present invention is designed to accurately infiltrate or infuse fluid over a wide area immediately surrounding a defined tract within a patient's body. The present invention may be utilized for additional medical applications including, but not limited to, delivering thrombolytic medication to a specific blood clot site within a blood vessel or delivering occluding agents to a tumor or bleeding vessel.

### BRIEF DESCRIPTION OF THE DRAWINGS

FIG. 1A is a perspective view of the present invention.

FIG. 1B is a perspective view of the tubular infusion member.

FIG. 2 is a perspective view of the tubular sleeve member.

FIG. 3 is an exploded perspective view of the present invention.

FIG. 4 is a front internal view of the present invention.

FIG. 5 is a detail view of the present invention taken along circle A of FIG. 4.

FIG. 6 is a schematic cross-sectional view of the present invention depicting the hydraulic seal in between the tubular infusion member and the tubular sleeve member.

FIG. 7 is a perspective view of an alternative embodiment of the present invention in which the tubular infusion member is able to protrude from the tubular sleeve member.

FIG. 8 is a perspective view of the present invention in which the infusion lip is resting upon the sleeve lip.

### DETAIL DESCRIPTIONS OF THE INVENTION

All illustrations of the drawings are for the purpose of describing selected versions of the present invention and are not intended to limit the scope of the present invention.

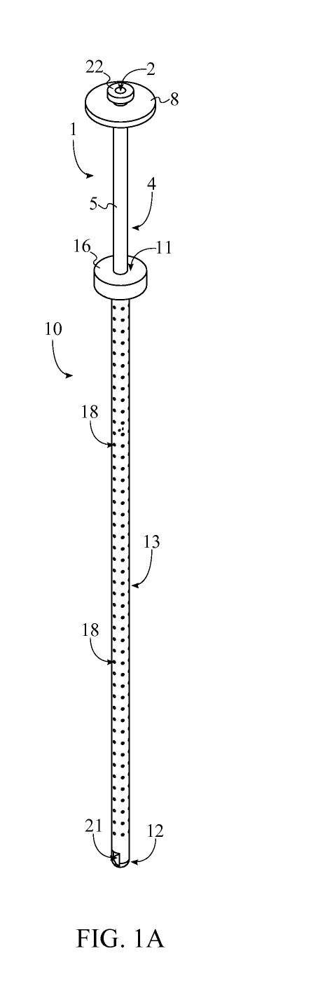

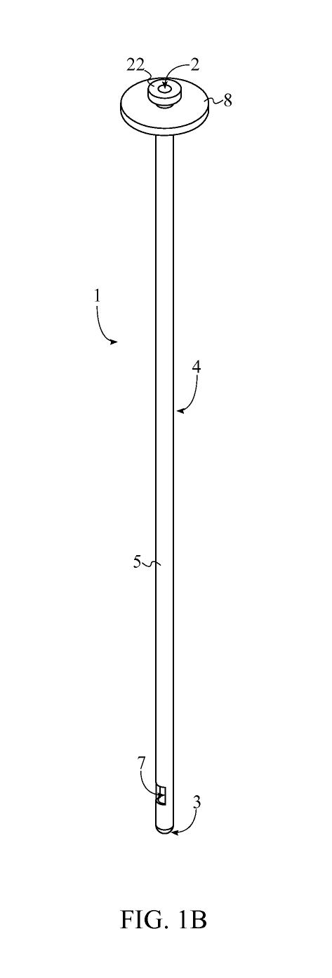

The present invention is a multiple side hole infusion apparatus that increases the effectiveness of a fluid such as a local anesthetic that is infiltrated or infused into deep tissues by distributing the fluid to a wider area surrounding an operation site in addition to other medical applications. With reference to FIG. 1A, the present invention comprises a tubular infusion member 1, a tubular sleeve member 10, and a plurality of fluid-distribution ports 18. The tubular infusion member 1 is slidably inserted into the tubular sleeve member 10 while medical fluid is able to exit the tubular sleeve member 10 through the plurality of fluid-distribution ports 18.

With reference to FIG. 1B, the tubular infusion member 1 is the elongated hollow member that is inserted into the tubular sleeve member 10. The tubular infusion member 1 may be a needle, a catheter, or a similar medical device. The tubular infusion member 1 is the component that holds and delivers medical fluid to the tubular sleeve member 10. The tubular infusion member 1 comprises an infusion open end 2, an infusion rounded closed end 3, an infusion tubular body 4, and a fluid-release port 7. The infusion open end 2 is the end that allows the medical fluid to be delivered into the tubular infusion member 1. The infusion rounded closed end 3 is the end of the tubular infusion member 1 that is inserted into the tubular sleeve member 10. The infusion tubular body 4 is the portion of the tubular infusion member 1 that is able to coaxially slide within the tubular sleeve member 10. The fluid-release port 7 allows medical fluid within the tubular infusion member 1 to exit the tubular infusion member 1. The fluid-release port 7 laterally traverses into the infusion tubular body 4. As such the medical fluid is able to pass from within the infusion tubular body 4 through the fluid-release port 7. The fluid-release port 7 is offset from the infusion rounded closed end 3 along the infusion tubular body 4. This allows the medical fluid to infiltrate along the length of the tubular sleeve member 10 as well as directly at the target area within the patient's body.

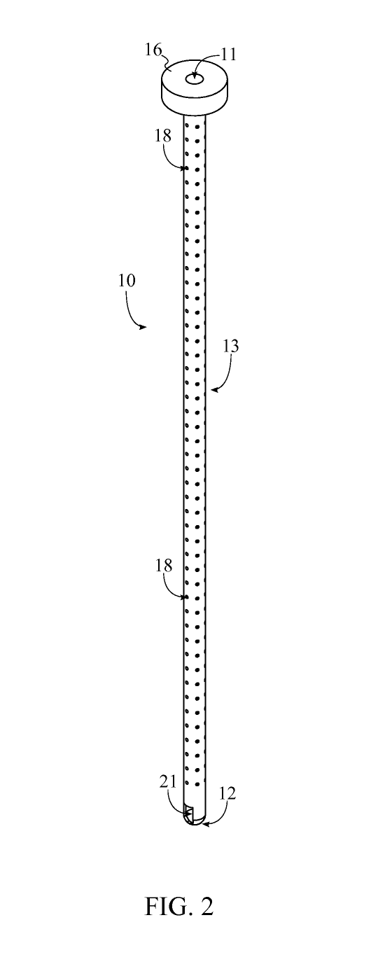

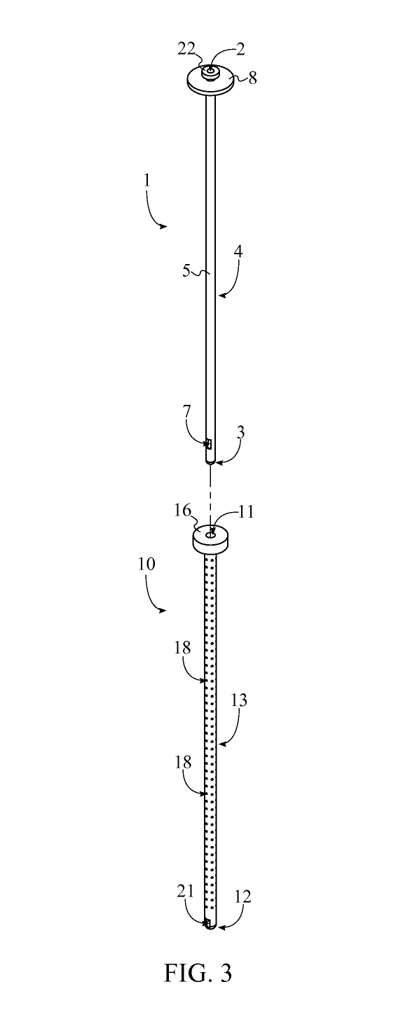

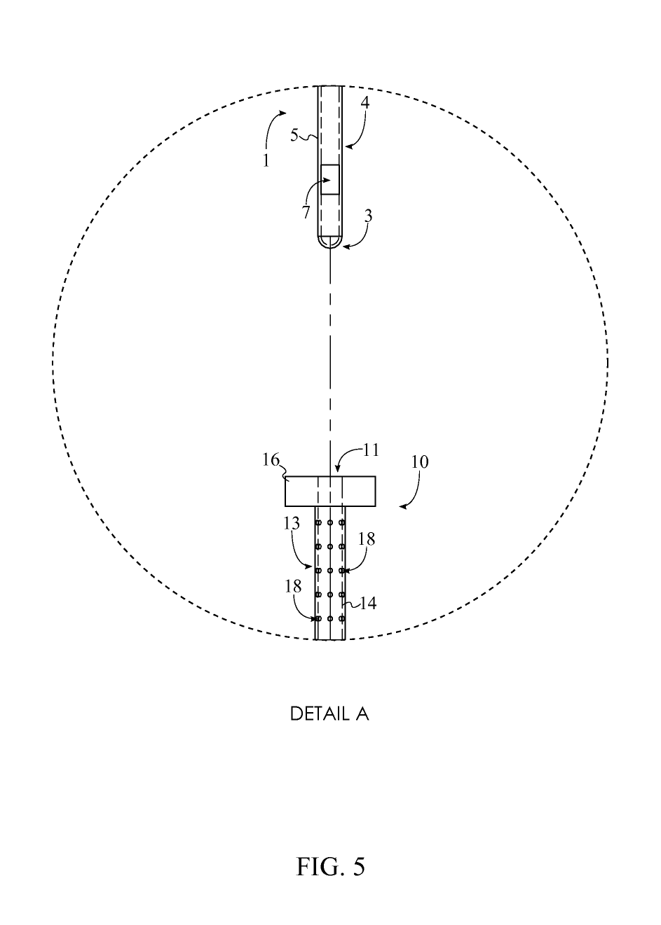

The tubular sleeve member 10 is the elongated hollow member that sleeves the tubular infusion member 1. Additionally, the tubular sleeve member 10 is the component that distributes the medical fluid from the tubular infusion member 1 into the desired area within a medical patient's body. The tubular sleeve member 10 is shown in FIG. 2 and comprises a sleeve open end 11, a sleeve closed end 12, and a sleeve tubular body 13. The sleeve open end 11 is the end of the tubular sleeve member 10 into which the tubular infusion member 1 is inserted during use of the present invention as shown in FIGS. 3-5. The infusion tubular body 4 is telescopically engaged to the sleeve tubular body 13 at the sleeve open end 11. This allows the infusion tubular body 4 to be inserted into and removed from the sleeve tubular body 13. The sleeve closed end 12 and the sleeve open end 11 are opposing ends of the tubular sleeve member 10. The sleeve closed end 12 may be blunted in catheter applications or sharpened/beveled for needle applications.

The sleeve tubular body 13 is the portion of the tubular sleeve member 10 along which the medical fluid within the tubular infusion member 1 is distributed. The plurality of fluid-distribution ports 18 laterally traverses through the sleeve tubular body 13. This allows the medical fluid from the tubular infusion member 1 to exit from within the tubular infusion member 1 and the tubular sleeve member 10 through the plurality of fluid-distribution ports 18. The plurality of fluid-distribution ports 18 is distributed along the sleeve tubular body 13. Additionally, the sleeve tubular body 13 is in fluid communication with the plurality of fluid-distribution ports 18 through the fluid-release port 7. As such, the plurality of fluid-distribution ports 18 and the fluid-release port 7 allow the medical fluid within the tubular infusion member 1 to be distributed along a length 15 of the sleeve tubular body 13. This increases the distribution region of the medical fluid within the patient's body.

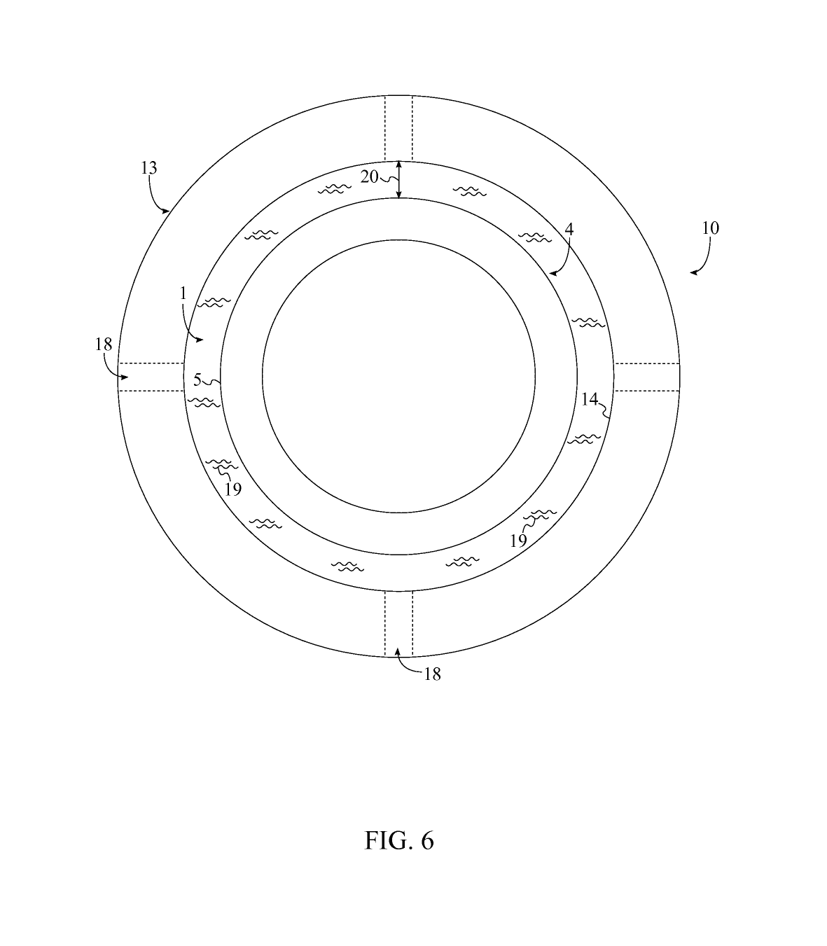

The present invention further comprises a quantity of dynamic fluid 19. The quantity of dynamic fluid 19 is the medical fluid that is held within the infusion tubular body 4 prior to distribution through the plurality of fluid-distribution ports 18. As shown in FIG. 6, an inner lateral surface 14 of the sleeve tubular body 13 is radially offset from an outer lateral surface 5 of the infusion tubular body 4 by an annular clearance 20. The annular clearance 20 is a space in between the sleeve tubular body 13 and the infusion tubular body 4 into which the quantity of dynamic fluid 19 is able to enter after exiting from within the infusion tubular body 4 through the fluid-release port 7. The quantity of dynamic fluid 19 is located within the annular clearance 20 in order to allow the quantity of dynamic fluid 19 to pass through the plurality of fluid-distribution ports 18 after exiting through the fluid-release port 7. The quantity of dynamic fluid 19 forms a hydraulic seal between the inner lateral surface 14 and the outer lateral surface 5. The hydraulic seal allows the tubular infusion member 1 to closely slide within the tubular sleeve member 10. Additionally, the hydraulic seal allows the quantity of dynamic fluid 19 to exit along the length of the tubular sleeve member 10 through the plurality of fluid-distribution ports 18.

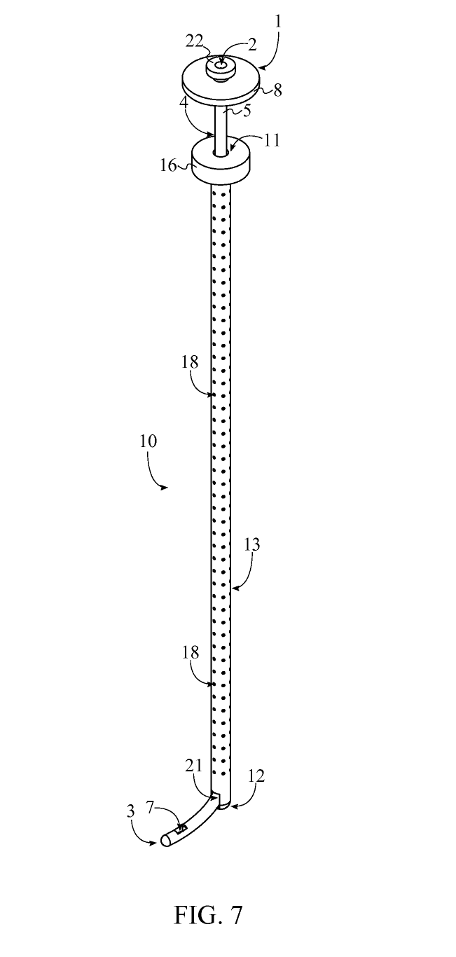

The present invention further comprises a guide port 21 as shown in FIGS. 2-4 and FIG. 8. The guide port 21 may be utilized in medical applications such as, but not limited to, inserting a guide wire in anticipation of inserting a cannula or catheter to remove fluid or air at an operating site. The guide port 21 laterally traverses into the sleeve tubular body 13, allowing a guide wire or similar object to be inserted into the sleeve open end 11, through the sleeve tubular body 13, and out through the guide port 21. The guide port 21 is positioned adjacent to the sleeve closed end 12. This allows the guide wire or similar object to exit the sleeve tubular body 13 adjacent to the sleeve closed end 12. In the alternative embodiment of the present invention shown in FIG. 7, the infusion tubular body 4 is made of a flexible material while the sleeve tubular body 13 is made of a rigid material. In this alternative embodiment, the rigid material of the sleeve tubular body 13 allows the sleeve tubular body 13 to support the flexible material of the infusion tubular body 4. Additionally, in this alternative embodiment of the present invention, a length 6 of the infusion tubular body 4 is greater than the length 15 of the sleeve tubular body 13. This allows the tubular infusion member 1 to extend from the tubular sleeve member 10 adjacent to the sleeve closed end 12. The infusion tubular body 4 traverses through the guide port 21, allowing the infusion tubular body 4 to protrude from the sleeve tubular body 13 through the guide port 21. This alternative embodiment of the present invention allows the user to insert a guide wire or similar object through the tubular infusion member 1 and establish access to an operating site through the fluid-release port 7. Once the guide wire is in place, the user is then able to remove both the tubular infusion member 1 and the tubular sleeve member 10.

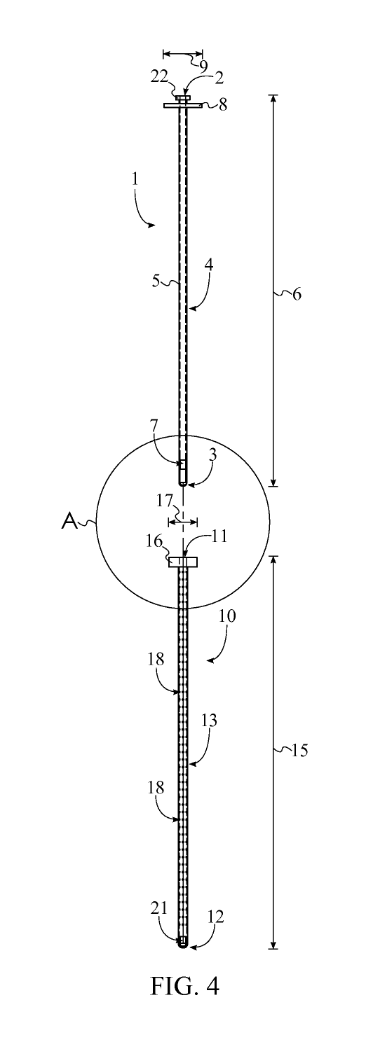

With reference to FIG. 1A, FIG. 3, and FIG. 4, the present invention further comprises a fluid infusion port 22 that is used to input the quantity of dynamic fluid 19 that is to be distributed through the plurality of fluid-distribution ports 18. The fluid infusion port 22 is adjacently connected to the infusion open end 2. As such, the quantity of dynamic fluid 19 is able to enter into the infusion tubular body 4 from the fluid infusion port 22. The fluid infusion port 22 is in fluid communication with the fluid-release port 7 through the infusion open end 2 and through the infusion tubular body 4.

This allows the quantity of dynamic fluid 19 to enter the infusion tubular body 4 through the infusion open end 2 before exiting through the fluid-release port 7. Additionally, the fluid-release port 7 is in fluid communication with the plurality of fluid-distribution ports 18 through the sleeve tubular body 13. After the quantity of dynamic fluid 19 exits through the fluid-release port 7, the quantity of dynamic fluid 19 travels through the sleeve tubular body 13 before exiting through the plurality of fluid-distribution ports 18.

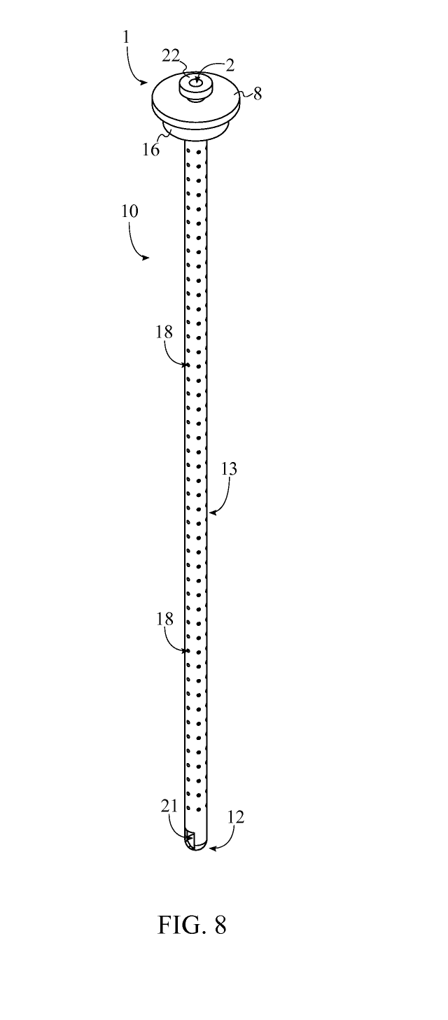

Again with reference to FIG. 1A, FIG. 1B, and FIGS. 2-5, the tubular infusion member 1 further comprises an infusion lip 8 and the tubular sleeve member 10 further comprises a sleeve lip 16. The infusion lip 8 prevents the tubular infusion member 1 from falling into the tubular sleeve member 10. The sleeve lip 16 is the surface upon which the infusion lip 8 is able to rest in order to prevent the tubular infusion member 1 from falling into the tubular sleeve member 10. The infusion lip 8 is connected around the infusion tubular body 4, adjacent to the infusion open end 2. Additionally, the sleeve lip 16 is connected around sleeve tubular body 13, adjacent to the sleeve open end 11. This allows the majority of the infusion tubular body 4 to enter into the sleeve tubular body 13 before the infusion lip 8 comes into contact with the sleeve lip 16, preventing the infusion tubular body 4 from falling fully into the sleeve tubular body 13. As shown in FIG. 8, a diameter 9 of the infusion lip 8 is greater than a diameter 17 of the sleeve lip 16, allowing the infusion lip 8 to rest upon the sleeve lip 16 and preventing the infusion tubular body 4 from falling into the sleeve tubular body 13.

Although the present invention has been explained in relation to its preferred embodiment, it is understood that many other possible modifications and variations can be made without departing from the spirit and scope of the present invention as hereinafter claimed.

What is claimed is:

1. A multiple side hole infusion apparatus comprises:
a tubular infusion member;
a tubular sleeve member;
a plurality of fluid-distribution ports;
the tubular infusion member comprises an infusion open end, an infusion rounded closed end, an infusion tubular body, and a fluid-release port;
the tubular sleeve member comprises a sleeve open end, a rounded closed end, and a sleeve tubular body;
the fluid-release port laterally traversing into the infusion tubular body;
the fluid-release port being offset from the infusion rounded closed end along the infusion tubular body;
the infusion tubular body being telescopically engaged to the sleeve tubular body at the sleeve open end;
the plurality of fluid-distribution ports laterally traversing through the sleeve tubular body;
the plurality of fluid-distribution ports being distributed along the sleeve tubular body; and
the sleeve tubular body being in fluid communication with the plurality of fluid-distribution ports through the fluid-release port.

2. The multiple side hole infusion apparatus as claimed in claim 1 further comprises:
a quantity of dynamic fluid;
an inner lateral surface of the sleeve tubular body being radially offset from an outer lateral surface of the infusion tubular body by an annular clearance;
the quantity of dynamic fluid being located within the annular clearance; and
the quantity of dynamic fluid forming a hydraulic seal between the inner lateral surface and the outer lateral surface.

3. The multiple side hole infusion apparatus as claimed in claim 1 further comprises:
a guide port;
the guide port laterally traversing into the sleeve tubular body; and
the guide port being positioned adjacent to the sleeve closed end.

4. The multiple side hole infusion apparatus as claimed in claim 3 further comprises:
the infusion tubular body being made of a flexible material;
the sleeve tubular body being made of a rigid material;
a length of the infusion tubular body being greater than a length of the sleeve tubular body; and
the infusion tubular body traversing through the guide port.

5. The multiple side hole infusion apparatus as claimed in claim 1 further comprises:
a fluid infusion port;
the fluid infusion port being adjacently connected to the infusion open end;
the fluid infusion port being in fluid communication with the fluid-release port through the infusion open end and through the infusion tubular body; and
the fluid-release port being in fluid communication with the plurality of fluid-distribution ports through the sleeve tubular body.

6. The multiple side hole infusion apparatus as claimed in claim 1 further comprises:
the tubular infusion member further comprises an infusion lip;
the tubular sleeve member further comprises a sleeve lip;
the infusion lip being connected around the infusion tubular body, adjacent to the infusion open end;
the sleeve lip being connected around the sleeve tubular body, adjacent to the sleeve open end; and
a diameter of the infusion lip being greater than a diameter of the sleeve lip.

7. A multiple side hole infusion apparatus comprises:
a tubular infusion member;
a tubular sleeve member;
a plurality of fluid-distribution ports;
a guide port;
the tubular infusion member comprises an infusion open end, an infusion rounded closed end, an infusion tubular body, and a fluid-release port;
the tubular sleeve member comprises a sleeve open end, a sleeve closed end, and a sleeve tubular body;
the fluid-release port laterally traversing into the infusion tubular body;
the fluid-release port being offset from the infusion rounded closed end along the infusion tubular body;
the infusion tubular body being telescopically engaged to the sleeve tubular body at the sleeve open end;
the plurality of fluid-distribution ports laterally traversing through the sleeve tubular body;
the plurality of fluid-distribution ports being distributed along the sleeve tubular body;
the sleeve tubular body being in fluid communication with the plurality of fluid-distribution ports through the fluid-release port;
the guide port laterally traversing into the sleeve tubular body; and
the guide port being positioned adjacent to the sleeve closed end.

8. The multiple side hole infusion apparatus as claimed in claim 7 further comprises:
a quantity of dynamic fluid;
an inner lateral surface of the sleeve tubular body being radially offset from an outer lateral surface of the infusion tubular body by an annular clearance;
the quantity of dynamic fluid being located within the annular clearance; and
the quantity of dynamic fluid forming a hydraulic seal between the inner lateral surface and the outer lateral surface.

9. The multiple side hole infusion apparatus as claimed in claim 7 further comprises:
the infusion tubular body being made of a flexible material;
the sleeve tubular body being made of a rigid material;
a length of the infusion tubular body being greater than a length of the sleeve tubular body; and
the infusion tubular body traversing through the guide port.

10. The multiple side hole infusion apparatus as claimed in claim 7 further comprises:
a fluid infusion port;
the fluid infusion port being adjacently connected to the infusion open end;
the fluid infusion port being in fluid communication with the fluid-release port through the infusion open end and through the infusion tubular body; and
the fluid-release port being in fluid communication with the plurality of fluid-distribution ports through the sleeve tubular body.

11. The multiple side hole infusion apparatus as claimed in claim 7 further comprises:
the tubular infusion member further comprises an infusion lip;
the tubular sleeve member further comprises a sleeve lip;
the infusion lip being connected around the infusion tubular body, adjacent to the infusion open end;
the sleeve lip being connected around the sleeve tubular body, adjacent to the sleeve open end; and
a diameter of the infusion lip being greater than a diameter of the sleeve lip.

12. A multiple side hole infusion apparatus comprises:
a tubular infusion member;
a tubular sleeve member;
a plurality of fluid-distribution ports;
a fluid infusion port;
the tubular infusion member comprises an infusion open end, an infusion rounded closed end, an infusion tubular body, and a fluid-release port;
the tubular sleeve member comprises a sleeve open end, a sleeve closed end, and a sleeve tubular body;
the fluid-release port laterally traversing into the infusion tubular body;
the fluid-release port being offset from the infusion rounded closed end along the infusion tubular body;
the infusion tubular body being telescopically engaged to the sleeve tubular body at the sleeve open end;
the plurality of fluid-distribution ports laterally traversing through the sleeve tubular body;
the plurality of fluid-distribution ports being distributed along the sleeve tubular body;
the sleeve tubular body being in fluid communication with the plurality of fluid-distribution ports through the fluid-release port;
the fluid infusion port being adjacently connected to the infusion open end;
the fluid infusion port being in fluid communication with the fluid-release port through the infusion open end and through the infusion tubular body; and
the fluid-release port being in fluid communication with the plurality of fluid-distribution ports through the sleeve tubular body.

13. The multiple side hole infusion apparatus as claimed in claim 12 further comprises:
a quantity of dynamic fluid;
an inner lateral surface of the sleeve tubular body being radially offset from an outer lateral surface of the infusion tubular body by an annular clearance;
the quantity of dynamic fluid being located within the annular clearance; and
the quantity of dynamic fluid forming a hydraulic seal between the inner lateral surface and the outer lateral surface.

14. The multiple side hole infusion apparatus as claimed in claim 12 further comprises:
a guide port;
the guide port laterally traversing into the sleeve tubular body; and
the guide port being positioned adjacent to the sleeve closed end.

15. The multiple side hole infusion apparatus as claimed in claim 14 further comprises:
the infusion tubular body being made of a flexible material;
the sleeve tubular body being made of a rigid material;
a length of the infusion tubular body being greater than a length of the sleeve tubular body; and
the infusion tubular body traversing through the guide port.

16. The multiple side hole infusion apparatus as claimed in claim 12 further comprises:
the tubular infusion member further comprises an infusion lip;
the tubular sleeve member further comprises a sleeve lip;
the infusion lip being connected around the infusion tubular body, adjacent to the infusion open end;
the sleeve lip being connected around the sleeve tubular body, adjacent to the sleeve open end; and
a diameter of the infusion lip being greater than a diameter of the sleeve lip.
